(19) United States
(12) Patent Application Publication
(10) Pub. No.: US 2011/0299036 A1
(43) Pub. Date: Dec. 8, 2011

(54) PORTABLE DIGITAL DIRECT OPHTHALMOSCOPE

(76) Inventor: Daniel M. Goldenholz, Sacramento, CA (US)

(21) Appl. No.: 13/152,113

(22) Filed: Jun. 2, 2011

Related U.S. Application Data
(60) Provisional application No. 61/350,742, filed on Jun. 2, 2010.

(51) Int. Cl. A61B 3/12; A61B 3/14

(52) U.S. Cl. 351/206

(57) ABSTRACT

A portable digital ophthalmoscope includes standard ophthalmoscope optics for viewing retinal images and a digital image sensor is optically coupled to the optics for providing a retinal image to a digital display and/or a storage device.

This application claims the benefit of U.S. provisional patent application No. 61/350,742, filed Jun. 2, 2010, which is hereby incorporated herein by reference.

### BACKGROUND

The invention relates generally to apparatus and methods used in connection with portable, direct ophthalmoscopes.

An ophthalmoscope is a medical instrument used by physicians and others for examining the interior of the eye. It includes a light, a mirror with a single aperture through which the examiner views. It typically supports on a wheel multiple lenses of varying strengths, for magnifying the image being viewed by the examiner. A lens to be used for viewing is selected and moved into the optical axis of the device by turning the wheel.

A direct ophthalmoscope produces an upright, or unreversed, image of the retina, with a magnification of approximately 15 times. A physician typically uses a direct ophthalmoscope to inspect the fundus of the eye. Direct ophthalmoscopes are used by physicians all over the world. They are portable, relatively lightweight, and relatively inexpensive. They can be used to diagnose a variety of conditions, ranging from mild to life-threatening. These devices have changed very little over the years. Physician training with direct ophthalmoscopes is not emphasized in modern American medical schools and many physicians are poorly equipped to perform even a basic ophthalmologic exam because of this. One reason for this is that the exam is difficult to perform under typical circumstances: pupils of the eye are not dilated, and non-ophthalmologists rarely interact with patients that have dilated pupils. Dilated pupils are preferred.

Due of the nature of the device, there is no way to record and subsequently review what was seen through the device. However, a direct ophthalmoscope produced by Welch Allyn, Inc. includes a beamsplitter that sent part of the light to an optical viewfinder, and the other part to an attached camera. However, the device was found to be of high cost, and exhibited low light capture.

### SUMMARY

Examples of a portable, direct ophthalmoscope described below optically couple an optical system that is part of a typical ophthalmoscope to an image sensor for capturing retinal images.

In one exemplary embodiment a digital image capture device, such as a digital camera, with a display is coupled to a standard, portable direct ophthalmoscope in a manner that focuses the image of a patient's retina coming through the ophthalmoscope onto an image sensor within the image capture device.

In a second exemplary embodiment, an image sensor and an image processor are directly coupled into the head of the ophthalmoscope. A display mounted to the back of ophthalmoscope displays the image from the image processor.

In each of the two exemplary embodiments, a clinician uses the display from the back of the camera to position the entire device, and can then record still images from the camera once focused on the retina, or record video of parts or the entire ophthalmologic exam.

In each of the two exemplary embodiments, none of the light being collected from the ophthalmoscope is split between an image sensor, whether it is part of or integrated with the ophthalmoscope or is part of a separate image capture device that is attached to the ophthalmoscope. All of the light being reflected from the patient's retina and passing through the focusing optics is coupled with an image sensor for ultimate display. By not splitting the light passing through the optics of the ophthalmoscope between an optical viewfinder and an image sensor, which reduces the light levels to each, all of the light is transmitted to the image sensor for enabling optimal image quality for producing a processed image for use by a clinician or diagnostician.

### BRIEF DESCRIPTION OF THE DRAWINGS

FIG. 1 is schematic illustration of an ophthalmoscope optically coupled with an image capture device.

FIG. 2 is schematic illustration of an ophthalmoscope having an integrated image sensor optically coupled with the optical system of the ophthalmoscope.

FIG. 3 is a flow chart representing an autofocus process for an ophthalmoscope.

FIG. 4 is a flow chart representing a process for automated retina detection and diagnostic processing.

FIG. 5 is a flow chart representing a process for measuring venous or arterial pulsations present in a retina.

FIG. 6 is a flow chart representing automated spherical aberration detection.

FIG. 7 is a schematic diagram of communication links between an ophthalmoscope, such as the ones of FIGS. 1 and 2, and remote devices capable of displaying images.

FIG. 8 is a flow chart representing a process for handling digital images generated by the ophthalmoscopes of FIGS. 1 and 2.

### DETAILED DESCRIPTION OF EXEMPLARY EMBODIMENTS

In the following description, like numbers refer to like elements.

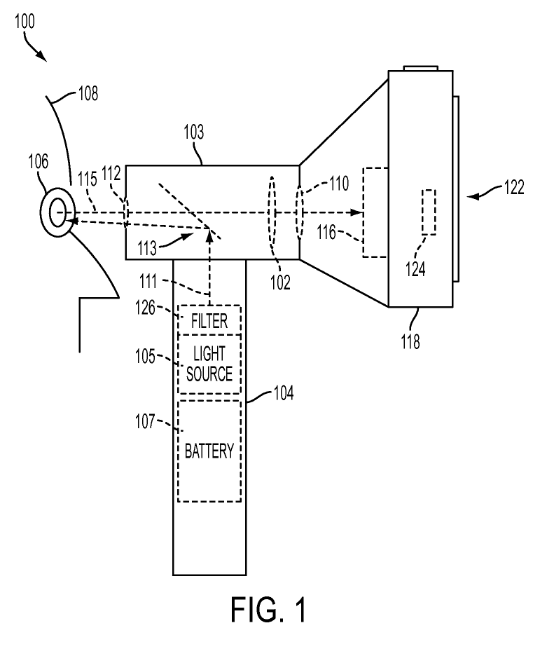

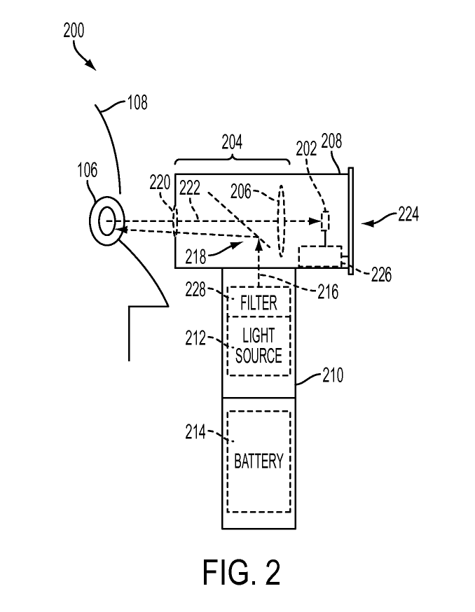

FIGS. 1 and 2 illustrate two different examples of portable direct ophthalmoscopes in which a digital image sensor is coupled to the optics of the ophthalmoscope to capture retinal images. In the embodiment of FIG. 1, a digital image capture device with a display is coupled to a standard, portable direct ophthalmoscope. The embodiment of FIG. 2 incorporates an image sensor and an image processor directly into the head of the ophthalmoscope. A display mounted to the back of ophthalmoscope displays the image from the image processor. The ophthalmoscope of FIG. 2 is relatively smaller, but does not have a standard, optical viewfinder. In each case, a clinician uses the display from the back of the camera to position the entire device, and can then record still images from the camera once focused on the retina, or record video of parts or the entire ophthalmologic exam.

In each of the illustrated examples it is preferred that none of the light being collected by the ophthalmoscope be split between an image sensor, whether it is part of or integrated with the ophthalmoscope or is part of a separate image capture device that is attached to the ophthalmoscope. All of the light being reflected from the patient's retina and passing through the focusing optics is coupled with an image sensor for ultimate display. By not splitting the light passing through the optics of the ophthalmoscope between an optical viewfinder and an image sensor, which reduces the light levels to each, all of the light is transmitted to the image sensor for enabling optimal image quality for producing a processed image for use by a clinician or diagnostician.

Referring now to FIG. 1, a portable, handheld direct ophthalmoscope 100 is comprised of a set of ophthalmoscope optics, which are schematically represented in the drawing and designated by reference number 102, disposed within a housing 103, through which to view the retina of an eye 106 of patient 108 undergoing examination. The optics 102, housing 103, and other elements located within the housing 103 are generally referred to as the “head” of the ophthalmoscope. An ophthalmoscope includes a handle 104 for enabling a physician or technician to maneuver and hold the ophthalmoscope in a position to view the retina of the eye through the eye's pupil. A light source 105 for illuminating the retina and a battery 107 for powering the light source is, in the example illustrated, placed inside the handle. The light source could, however, be powered by line current or other means.

One example of a portable, direct ophthalmoscope is a PANOPTIC® ophthalmoscope made by Welch Allyn Inc. Other types of conventional direct ophthalmoscope heads could be adapted for use.

The conventional operation of an ophthalmoscope involves a physician, technician or other user viewing inside the eye of the patient through an optical viewfinder 110, which is comprised of a rear aperture formed on the back end of housing 103 that contains a lens, which is aligned with optical system 102. The opposite end of the optical system is placed near the eye of the patient. The retina is illuminated by light from the light source. In this example of an ophthalmoscope, a beam of light in from the light source is bounced by a tilted mirror 113 and directed through forward aperture 112 into the eye through its pupil. Light reflected by the retina, generally indicated by line 115, is received through forward aperture 112, which, in this example, holds a lens, and is then collected and focused by optical system 102 for viewing through the viewfinder 110. A hole in the titled mirror 113 permits light 115 to pass the mirror.

In other examples of ophthalmoscopes, the beam of light for illuminating the retina passes through an aperture separate from the forward aperture 112, through which light reflecting from the retina passes The light source may also be located in the head.

The optical system and illumination system are representative only and have been schematically illustrated. Although the illustrated optical system has an axis aligned with the forward aperture and rear aperture, the light passing through optical system can be reflected using mirrors if the forward and rear apertures are not located along a common axis.

In the embodiment illustrated by FIG. 1, a lens 116 of a camera 118 is held adjacent to the viewfinder no of the head by connector 120. Connector no acts to hold the camera 118 at the correct location, so that the lens 116 of the camera focuses light from the optical system 102, exiting viewfinder no, onto the camera's image sensor. Camera 118 is an example of a self-contained image capture device.

The camera 118 is, in the illustrated embodiment, a completely self—encapsulated digital camera, capable of displaying and capturing still and/or moving images. The body of the camera houses, for example, a CMOS, sCMOS, CCD or other type of digital image sensor, schematically represented by element 124, an image processor (not shown) for converting the signals from the image sensor into a digital image for display or recording. Lens 116 focuses light coming from the viewfinder of the optics 102 of the ophthalmoscopic head on to the image sensor.

In the illustrated embodiment, a user views an image of the retina of a patient's eye on a digital display 122 that is part of digital camera 118. In the illustrated embodiment, substantially all of the light passing through the ophthalmoscope optics 102 is provided to the image sensor without any light splitting in the optical path.

The digital camera displays processed images of the retina of the eye 106 of the patient 108 on display 122. The display may be integrated with, or otherwise affixed to, a back side of the camera, or mounted to the body of the camera by, for example, a hinge to the camera. The processor of the camera may also be configured to write the image to a memory card or other digital storage device within or attached to camera 118. The image capture device may, instead of, or in addition to, displaying and/or storing the images, include a wired or wireless interface (not illustrated in FIG. 1) for transmitting retinal images to another device, such as a computer (e.g. server, desktop, laptop, or tablet), smartphone, or a storage device, over a wireless and/or wired connection.

The camera 118 may be part of, or integrated with, for example, other relatively small, portable electronic devices. Types of image capture devices include commonly available digital photographic cameras (the Canon® Elf® or Fujifilm® Finepix® compact cameras, for instance, or larger DSLR cameras), various smart phones (an iPhone®, or Android operating system based phones, for instance) with built in cameras, digital video recording devices (such as the Flip® series from Cisco), or personal digital assistant devices (such as an iPod Touch® from Apple, Inc), tablet computers, and other such devices having an image sensor, a lens for focusing images onto the image sensor, and a processor for processing the images for display, storage or transmission, all within a protective housing or case.

The connector positions the image capture device at a position that enables the light from the viewfinder to be focused on an image sensor within the device. Directing the resulting captured images to a small inexpensive display screen (such as an LCD display device) would permit viewing without the need to split the image. Such a device could be very inexpensive.

The connector 110 could be made variously from metal, plastic, rubber, other materials, or a combination of materials. The connector permits the interchangeable attachment and detachment of an external image capture device or multiple types of such devices. In one embodiment, the connector's shape can be changed or adapted for different types of cameras and/or different types of image capture devices.

One advantage of the illustrated embodiment is that it permits the user to connect or disconnect one or more than one kind of image capture device and operate the ophthalmoscope without a camera. The connector includes one or more components, not shown, for releasably attaching the connector to the housing 103 and to the image capture device. Examples of such attachment components include a latch, a hook, hook and loop fasteners (such as Velcro®), pressure, suction, clamps, magnets, screws, and other fasteners. In the illustrated embodiment, at least a portion of connector 120 is comprised of a rigid structure for supporting the camera in a fixed relationship to the optical system 102, the structure extending between and attaching to head or handle of the ophthalmoscope and the image capture device. Furthermore, it is preferred, but not necessary, that the connector completely surrounds the optical viewfinder 110 and lens 116 of the camera in order to reduce or prevent ambient light from entering the lens of the camera.

An example of such a connector is one comprised of a rigid arm or bracket, with a length and geometry that is adjustable, which is clamped to the head or handle and attached to the bottom of a camera with a screw. The connector could be constructed only for connecting a particular camera to the ophthalmoscope, or it may be configured or adapted for permitting swapping in and out of different digital cameras in front of the viewfinder, thus permitting interchangeable camera types/features. By making the length and/or geometry of the bracket or other rigid structure comprising the connector adjustable, the position of the image capture devices' camera can be adjusted as necessary, allowing different image capture devices to be used. A covering, which can be a made of a flexible or rigid material, including cloth, rubber, plastic or metal, extending between the viewfinder and the camera could be used in conjunction with the bracket to comprise a connector that blocks ambient light.

Ophthalmoscope 100 includes one or more filters for the light source 105. The one or more filters are schematically represented by filter 126. If there is more than one filter, it can be, for example, manually selected using a dial. Uses for the one or more filters are described below.

Referring now to FIG. 2, illustrated schematically is a second, exemplary embodiment of a portable, direct ophthalmoscope 200 in which a digital image sensor 202 is optically coupled to ophthalmoscope optics 204. Like portable, handheld direct ophthalmoscope 100, ophthalmoscope is comprised of a set of ophthalmoscope optics, which are schematically represented in the drawing and designated by reference number 206, disposed within a housing 208, through which to view the retina of an eye 106 of patient 108 undergoing examination. The ophthalmoscope includes a handle 210 for enabling a physician or technician to maneuver and hold the ophthalmoscope in a position to view the retina of the eye through the eye's pupil. A light source 212 for illuminating the retina is placed within the head of the ophthalmoscope. A battery 214 for powering the light source is mounted or placed inside a removable portion of the handle.

The retina of eye 106 is illuminated by light from the light source 212. In this example, a beam of light 216 from the light source is bounced by a tilted mirror 218 and directed through aperture 220 into the eye through its pupil. Light reflected by the retina, generally indicated by line 222, is received through aperture 220, which includes in this example a lens, and is then collected and focused by optical system 206. A hole in the titled mirror 218 permits light 222 to pass the mirror. In other examples of ophthalmoscopes, the beam of light for illuminating the retina passes through an aperture separate from the light reflecting from the retina. The optical system and illumination system are representative only and have been schematically illustrated.

Unlike ophthalmoscope 100, digital image sensor 202 is located within the head of the ophthalmoscope, in this case within the head of the ophthalmoscope, and in this example, within housing 208. Mounted to the head is a display 224, which takes the place of a traditional viewfinder. A processed image of the retina captured by the image sensor can be displayed on the display device, or can be stored in memory in a removable storage device, or elsewhere before, during and/or after its display on the display. The display device could have a touch screen and/or off to the side buttons for control of the display and other functions of the ophthalmoscope 200, as desired. Like ophthalmoscope 100 of FIG. 2, ophthalmoscope 200 can include one or more filters for the light source 212. The one or more filters are schematically represented by filter 228. The filters can be separate, static filters that are manually selected using, for example, a dial. However, the filter could also be implemented using an electronic display screen, through which light passes, such as those used in projectors. Pixels can be set electronically, using programmed instructions, in predetermined patterns, with predetermined colors. Uses for the one or more filters are described below.

Referring now to FIGS. 1 and 2, each of the ophthalmoscopes 100 and 200 can be adapted for use in infrared ophthalmoscopy by having the light source emit infrared radiation and having an image sensor that is sensitive to the wavelengths of infrared radiation being used. The typical light source of an ophthalmoscope could be exchanged for an IR light source, or an IR filter could be added to the set of available filters 126 or 228. By adjusting the intensity of light being transmitted to the patient's eye, the scope would offer gradients between two distinct modes—passive IR viewing and active IR illumination. The optics may require modification for optimized transmission and focus of IR wavelengths (if not already optimized, depending on the type of optics used). If the image sensor is not sensitive to the IR wavelengths being used, an intermediate component that illuminates visible light in response to IR light (such as phosphors used on x-ray equipment) could be used.

The two IR modes could be used for methods of pathological evaluation of the retina and even hidden structures beneath the retinal surface. Particularly, an embodiment with IR illumination could be useful for evaluation of the vasculature of the retina with greater levels of detail, arterieral occlusions, and neoplasias (cancers). With proper component selection, it would be possible to include the infrared mode as one of multiple possible modes of operation, including visible light.

The ophthalmoscopes of FIGS. 1 and 2 could include optical and/or digital image stabilization systems. Optical stabilization techniques would need to be incorporated into the optics portion of the scope, and would decrease the effect of operator movement on the stability of the retinal image. Digital stabilization techniques would stabilize retinal images via software techniques at the expense of image resolution. By including one or both such systems in a digital ophthalmoscope, retinal exams could be performed faster and resulting images would be clearer.

A direct ophthalmoscope of the type illustrated by FIGS. 1 and 2 could also be adapted for acuity testing. Typically, acuity testing is done using a standardized chart such as a Snellen Chart at a standardized distance from the viewer. Using a filter (e.g. one the one or more filters 126 of FIG. 1 or 228 of FIG. 2) that the light source passes through prior to entering the optics of the scope, it would be possible to display images to the patient's eye. To make these viewings comfortable, the light level would be decreased below the level typically used for retinal exams. The image displayed would be any one of the standard visual acuity charts. Other eye tests including standardized color blindness charts could be optionally included as possible filter settings as well. The filters could be implemented either as static physical filters that are manually cycled through with a dial, or using a semi-transparent display system, such as an LCD screen used in projectors, that modifies the source light as it passes through.

Alternatively, a complete display system could be optionally selected with a switch that would manually divert the output of a small display to the main optics instead of the standard light. This small display could be designed to fit inside the scope (perhaps only 0.5 cm square). The images would be scaled and if needed also transposed properly so as to display upright and in the proper dimensions when presented to the retina directly. A digital ophthalmoscope with integrated display screen would permit a more advanced control of the possible screen images presented to the patient's retina for acuity and other vision tests.

Each of the exemplary ophthalmoscopes 100 and 200 can also include one or more processors, running one or more software programs, or hardware, such as application specific integrated circuits, for implementing various additional functions and features. For example, in ophthalmoscope 200, processor 226 is coupled to the image sensor and the display. Processor 226 is representative of one or more general purpose processors, one or more special purpose processors, or a combination hardware, firmware and/or software for controlling operation of the ophthalmoscope in still image, video capture, and review modes, as well as performing additional logic for implementing additional features described below. The image sensor and processor could be formed as one monolithic “chip” or integrated circuit. Coupled with the processor 226 is memory for storing software instructions and data.

For example, ophthalmoscope 100 or 200, or the image capture device 112 connected to ophthalmoscope 100, can be programmed for enabling image capture and other functions to be voice controlled so as to reduce the complexity of holding the device and making adjustments to the digital camera.

An automatic focus and capture process that triggers image capture when there is proper focus could also be programmed. It would permit the user to simply aim at the retina and allows the device to autofocus and record a picture when a retinal image is detected.

And yet another example is a retrospective retinal image capture system. If images are captured in video mode, a simple detection algorithm attempts to identify frames with clear retinal images and isolate them as still frames for storage and review immediately after the video is collected. The ophthalmoscopes could be further programmed for performing an automated preliminary diagnostics system that includes an image recognition algorithm that attempts to make preliminary diagnosis for one or more retinal conditions, examples of which include AV nicking, papilledema, pallor, normal retina.

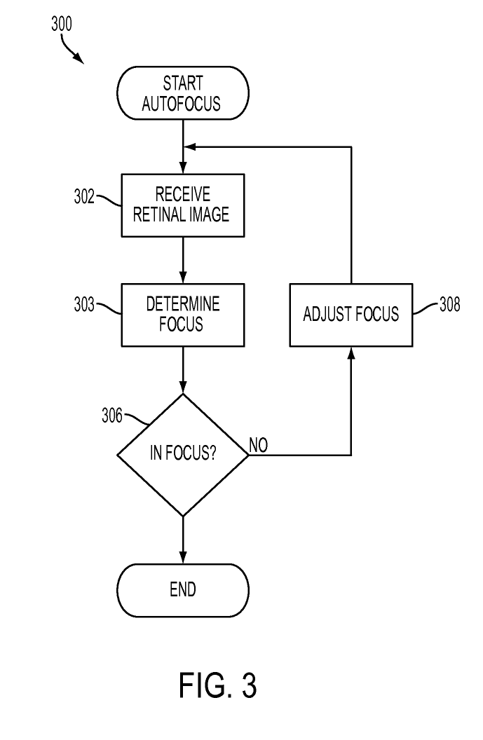

Referring now to FIG. 3, illustrated is a process 300 that implements a digital detection algorithm for determining the level of focus of an ophthalmoscope optically coupled with an image sensor, such as those of FIGS. 1 and 2. The process is performed by an autofocus circuit, which can be implemented using embedded processor in the ophthalmoscope or image capture device, running a software program, a hardware circuit, such as an application specific integrated circuit. At step 302, an image is received by the autofocus circuit. The focus of the image is determined at step 304. If the image is out of focus at step 306, the optics are adjusted (specifically the focal length adjusted) at step 308. The process is repeated until proper focus is achieved. There are multiple search algorithms that could be employed for determining focus and finding the correct focus, such as a simple incremental approach, or more sophisticated binary search techniques. A passive autofocus process and/or circuit of the type used in SLR digital cameras can be adapted for this purpose.

Alternately, a more sophisticated active autofocus system could be employed, that would take advantage of a directed pulse of infra-red light directed at the eye, bounced off and captured by an infra-red sensitive detector. Such a system would need to calculate the divergence between the expected focus and the detected focus, and adjust the focal length accordingly. A combination of passive and active systems could also be employed.

The autofocusing feature could also, in another embodiment, be used to automatically determine the need and amount of vision correction—the strength of glasses prescription, if any, needed by a patient correct for refraction errors. To do this, standard techniques used by autorefractors or aberrometers can be incorporated into the device. A beam of light is generated by the head of the ophthalmoscope. The could be done by, for example, filter (e.g. filter 228 of FIG. 2) that limits only a very small circle of light from a light source in the ophthalmoscope (e.g. Light source 212) in the center of the filter is used. The ophthalmoscope is equipped with a mechanism for controlling the focus of the beam of light. An autofocus process, preformed by an application specific integrated circuit or a program of stored instructions executing on a processor, for example, identifies the proper focal length required to focus the beam onto the retinal surface and determines from the focusing, or the focal length, the amount, if any, of correction for preparing prescription for glasses or contacts. This number can be output to the visual display and or by a small audio alert.

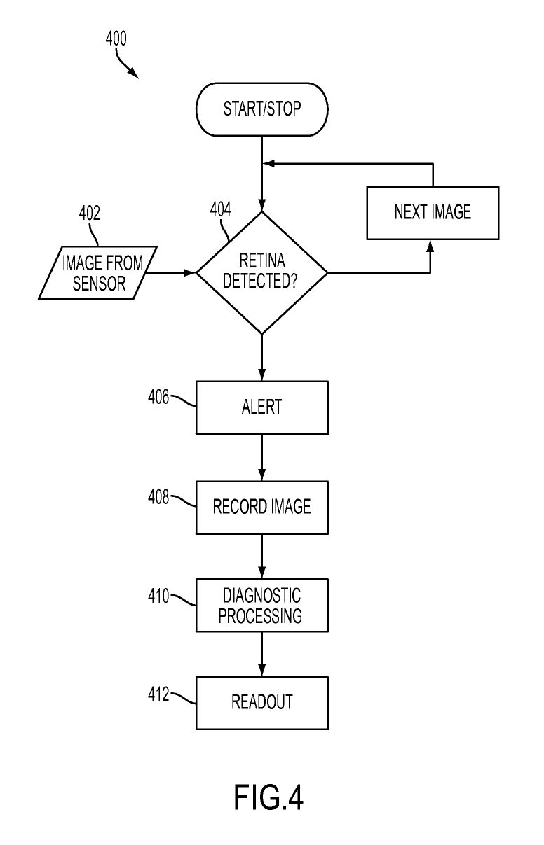

Referring now to FIG. 4, the schematic flow chart represents an example of a process 400 implemented by a hand-held, direct ophthalmoscope, with a digital image sensor, such as those of FIGS. 1 and 2. The process is implemented by, for example, special purpose logic circuitry, or by software or firmware, stored in memory, being executed by a general purpose processor or a special purpose processor, such as the image processor present in the examples given above.

The process permits the device to be used to both identify the retina once visualized, and further to diagnose and display information about it.

Capturing of series of images using an image sensor would be done as described above. Each image, as it is captured, is represented by image 402. That image is subject to a retina detection algorithm at step 404. The algorithm could employ one of a number of different techniques to determine that a retina is currently in view. Prior to detection, the images are preprocessed with spatial filters and other noise reduction techniques. The detection techniques could include, but are not limited to: neural networks, fuzzy logic, and statistical feature extraction.

If the likelihood of a retina detection exceeds a certain threshold, then an alert is set at step 406. This alert could take the form of an audible sound, such as a beep, click, or other user defined alert. It could also take the form of an audible voice that says a user-defined phrase, such as <<retina detected>>. Another type of alert could be visual—a small LED or other visual cue to alert the operator that a retina was identified and could be included on the digital capture system.

At step 408, the onset of retina detection could trigger image recording. In this way, recording of extraneous (non-retinal) images would be reduced and thus memory could be spared. In addition, only those images that are deemed likely to include a retina would then be fed into the diagnostic software module, which would then be used to review at step 410 the images and determine if the retina shows only healthy tissue, or evidence of disease states. A number of disease states could be identified by the diagnostic software, including but not limited to: AV nicking, Aicardi syndrome, Asteroid hyalosis, optic atrophy, branch retinal artery occlusion (BRAO), congenital hypertrophy of the retinal pigment epithelium (CHRPE), Best disease, bird shot, cataract, cat scratch, central retinal vein occlusion, choriodal neovascular membrane, choriodal nevus, cillio-retinal artery occlusion, macular edema, Coat's disease, Colomba, epiretinal membrane, familial exudative vitreoretinopathy, dundus albipunctata, drusen, giant retinal tear, gyrate atrophy, idiopathic polypoidal choroidal vasculopathy, intraocular foreign body, lattice retinal tear, retinal leukemia, macroaneursym, macular hole, melanocytoma, morning glory syndrome, myelinated nerve fiber layer, myopia lacquer checks, non-proliferative diabetic retinopathy, optic disc edema, optic nerve head drusen, persistent hyperplastic primary vitreous, pigment epithelial detachment, proliferative diabetic retinopathy, purtscher retinopathy, retinal hole, retinitis pigmentosa, rhegmatogenous retinal detachment, stargadt disease, toxoplasmosis. To detect these different pathological states, a number of possible algorithms could be employed, based on image segmentation, machine learning, fuzzy logic, neural networks, statistical feature extraction, and/or other techniques.

Finally, after a diagnosis is determined at step 410, a readout is given to the operator at step 412. This could be in the form of a digital text superimposed on the display, text displayed above/below/to the side of the image, or spoken feedback through a speaker. Diagnostic readouts could also be simplified to a very small series of possibilities, such as <<unable to diagnose>>, <<normal>>, <<abnormal>>. These three possibilities could then be read-out to the operator as above, or in the form of a colored LED attached to the digital capture element to simplify the display as described in previous figures.

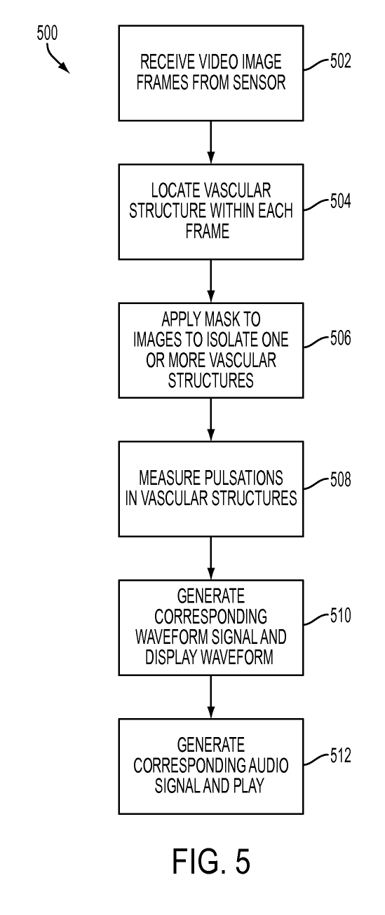

Referring now to FIG. 5, another feature for which the ophthalmoscopes 100 and 200 could be programmed or adapted offers an analysis tool for vascular function. In particular, process 500 evaluates the venous or arterial pulsations present in the retina, looking for abnormalities. Such a tool would have numerous possible applications, including functional assessment of intracranial vascular integrity, characterization of the degree of intracranial pressure in the case of idiopathic intracranial hypertension (pseudotumor cerebri), and assessment of vascular damage from atherosclerosis or vasculitis. The process would operate as follows.

The image sensor coupled with an ophthalmoscope, such as those of FIGS. 1 and 2, feed the images to a vessel detector process at step 504. This process would be optimized to locate vascular structures in the retina. It could use one of a number of techniques to do this, including spatial frequency filtering, color separation, cluster analysis (looking for functional signals that are temporally correlated), correlation analysis (looking for signals that correlate to externally measured cardiac pulsation signals), signal detection algorithms optimized for cardiac pulsation signals similar to those used in pulse-oximeter devices, neural networks, fuzzy logic, statistical feature analysis and so on.

After locating these structures, a mask is generated at step 506 that would cover all non-vascular structures from the image, and only vascular structures would be left in the image.

At step 508, spatially weighted averaging algorithms develop from the series of masked images a time domain one-dimensional signal. This signal may require noise reduction techniques such as low pass and high pass filtering. The signal could then be displayed either on a screen at step 510, or presented to the operator as an audible signal whose intensity varies with the strength of the signal at step 512.

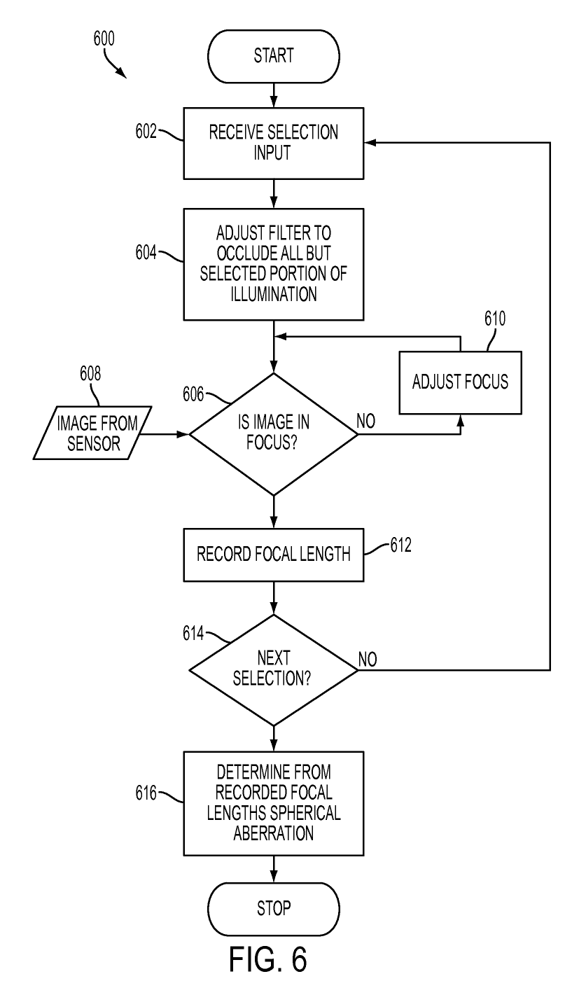

Referring now to FIG. 6, in an alternate embodiment, ophthalmoscope, such those of FIG. 1 or 2, is adapted for characterizing the spherical aberration, if present, in a given patient's eye using a process such as process 600.

To do this, the illuminating light (e.g. light from light sources 105 of FIG. 1 or 212 of FIG. 2) shines through a filter (107 of FIG. 1 or 228 of FIG. 2), which then goes through the optics. The filter is preferably designed to have multiple possible modes, which are selected at step 602. In one embodiment, the modes are selected by an automated control system in the head of the ophthalmoscope. Each mode occludes a portion of the light by adjusting the filter at step 604. If segmental regions of light are occluded, then various portions of the retinal image can be examined separately.

The optics 102 (FIG. 1) or 206 (FIG. 2) could similarly be controlled by an automated control system that includes a software controlled process and motors for moving or changing the elements of the optical system. At step 606, the image sensor provides images 608 to software-implemented algorithms that determine if the image is in focus. If not, the processor controls the optical system at step 610 to make adjustments until the appropriate level of focus is obtained, in a manner similar to that described above and similar to ‘passive’ autofocus techniques found on modern SLR cameras. The focal length or a representation of it is stored at step 612.

As indicated by step 614, this autofocus function is repeated for each filtered region of the retina. It would be expected that spherical aberrations would result in multiple focal lengths to be required to autofocus the different filtered segments. At step 616 the process determines whether a spherical aberration is present.

An alternative method to achieve the same outcome would avoid the sequential occlusion of light by the filter, but simply by using software by the digital capture system that ignores sequential portions of the retinal image. Either way, the system would be able to relatively quickly characterize spherical aberrations if they were present in the eye. Alternatively, active autofocus techniques could be used in combination with the above, or a combined active and passive autofocus system could be used. See the section on autofocus for more detail.

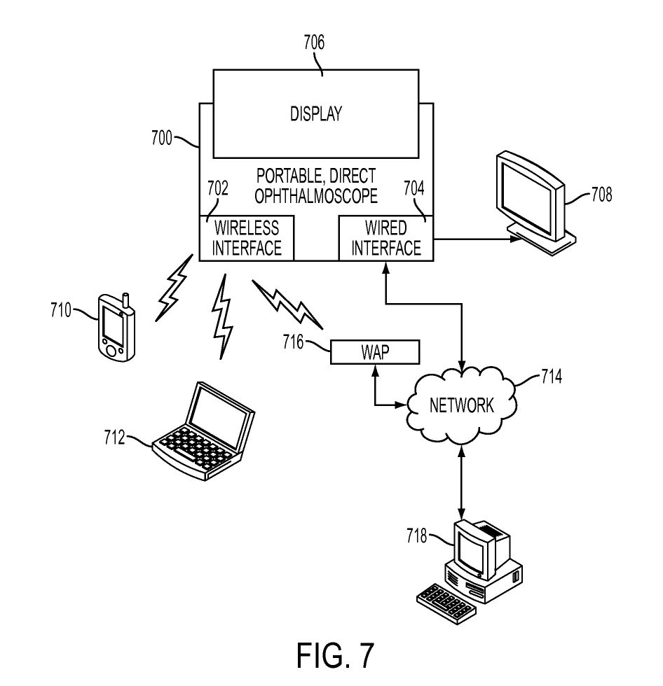

Referring to FIG. 7, a portable direct ophthalmoscope 700 is similar to ophthalmoscopes 100 and 200 of FIGS. 1 and 2. However, in addition to having an image sensor optically coupled with the optics of the ophthalmoscope (either incorporated into its head or into an image capture device connected to the head) it also includes one or more communication interfaces, which enable it to transmit imagery and other information to a local or a remote displays, computers and other devices. In the illustrated example, ophthalmoscope 700 includes one or more wireless interfaces 702 and one or more wired interfaces 704.

In this example, the ophthalmoscope also includes display 706, for example an LCD or OLED screen, mounted to the device. This display can be mounted directly on the head of the ophthalmoscope or on an image capture device connected to the viewfinder of the ophthalmoscope. The display is, preferably, as large as possible given the overall device size. For example, it preferably comprises the great majority of the back surface of the ophthalmoscope and has a resolution that matches the image capture system so as to display the maximal level of detail. Alternately, or in addition, the ophthalmoscope could incorporate a small projector that is able to project a larger image to a nearby surface. These systems are very small and lightweight. They would permit for a smaller portable design.

The ophthalmoscope 700 may, in place of or in addition to the display 706, transmit and display retinal images to another portable or fixed display, such as a smart phone, a tablet, laptop, desktop computer, or a monitor or projector, that is located within the examination room or remotely, and not directly attached to or incorporated into the ophthalmoscope. FIG. 7 illustrates several possibilities.

One example is a display 708 connected to the wired interface 704. The connection could take the form of a cable. Examples of interfaces include USB, Ethernet, VGA and/or HDMI interfaces that are connected to a television or large monitor, a projector (including pico or pocket sized projectors) or to a computer with a display screen. Other interfaces could include, for example, proprietary interfaces for smart phones, tablet computers and other similar devices. It could also comprise a USB port that enables connection to, for example, a personal computer or laptop.

Examples of wireless interface 702 include wireless Ethernet (“WiFi”), BlueTooth, or other wireless communication protocols. The wireless device that could receive such signals could be a nearby smartphone or tablet device, represented by device 910, a projector, personal computer 712, or receiver which could then connect to a standard TV, projector or monitor (not shown). Some newer TV models include WiFI interfaces that enable them to receive WiFi signals. If properly configured these devices could be used as well.

Network 714 represents one or more interconnected local area, wide area, or other networks, including the Internet. Using either the wired interface (such as an Ethernet interface) or wireless interface that connects to the network, for example, through a wireless access point or gateway 716, ophthalmoscope 700 could transmit retinal images and other information to a remote display or computer, represented by computer 718 in the figure.

Ophthalmoscope 700 could thus connect to a distant device—one not in the near proximity to the digital ophthalmoscope, possibly in a different room, different floor, different building, different city or country—that can store and/or display the images. Images could be sent via secured encrypted means over telephone lines or internet to remote viewing stations. This would permit the ability for multiple users at potentially multiple locations to simultaneously view the same images available to the operator locally.

For example, a technician operating the ophthalmoscope can be viewing the patient's eye with a display screen, which is simultaneously being transmitted (preferably by wireless secure communication means, but potentially by wired means) to a remote viewing station, which does not necessarily need to be in the same clinic/hospital.

The ophthalmoscope could be, optionally, equipped with audio two way communication capability, so that the remote viewer can give real time instructions to the operator. An example situation illustrates this concept. Rural health care center employs medical assistants to evaluate patients. As part of his basic evaluation, the assistant documents digital images of a patient's retinal exams. If there is something unusual about the images he obtains, and he has not seen findings like this before, he can initiate a real-time communication session with an expert, who directs the exam to include subtle features that the assistant may have missed, and evaluate the images. The assistant collects the requested images and records the results in the electronic medical record. If the patient has an abnormal condition that requires immediate attention, procedures for addressing the condition can be immediately scheduled and/or performed. Thus, abnormal-appearing retinal exams can be performed by a non-expert and medical decisions can be rapidly made thereby expediting the medical care of the patient. The remote viewing expert can review the images live on a large screen monitor, a projector, a tablet based device, or even a screen attached to a digital ophthalmoscope. They could be located in the same clinic/hospital, in a nearby central location, or in a very distant specialist outsourced group.

Finally, at either the same location or at a distant site, these digital images could be ‘displayed’ by printing the images onto paper or other human readable medium.

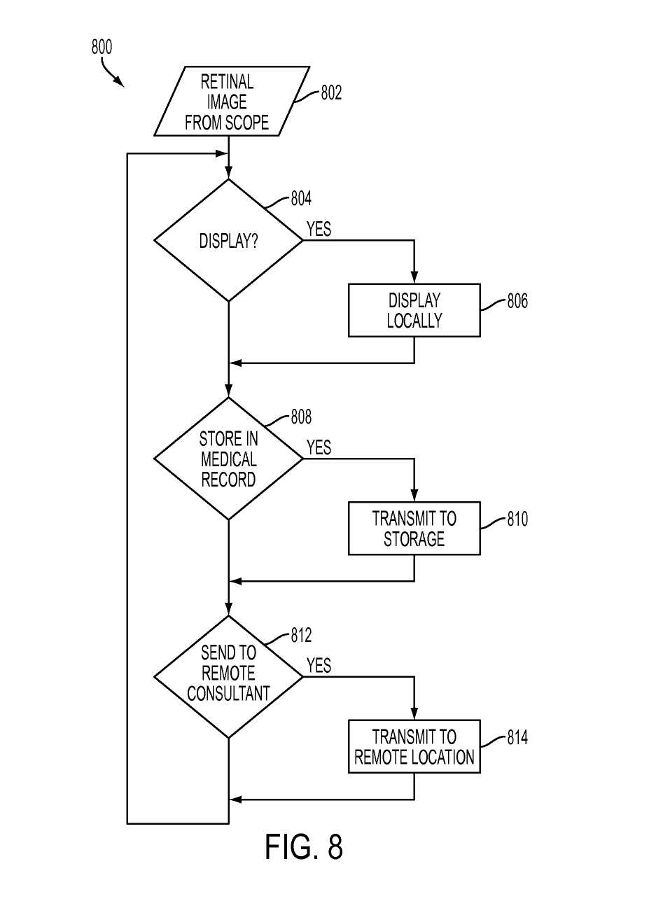

Referring now to FIG. 8, images 802 created by a digital image sensor from an ophthalmoscope such as those described in connection with FIGS. 1 and 2, could be used as part of a workflow illustrated by process 800. As represented by steps 804 and 806, each image could be presented immediately to the operator. However, the images could, instead, or in addition, be stored in a patient's medical record, depending on the mode of operation, as indicated by steps 808 and 810. In addition, the images could be transmitted digitally to a remote consultant, whose expertise exceeds the original operator, as indicated by steps 812 and 814.

For example, a minimally trained medical assistant operates a digital ophthalmoscope to examine the retina of patient. An ophthalmoscope can be programmed to automatically inform the assistant that he has correctly located a good view of the retina, and record images. These images are then transmitted to the patient's permanent electronic medical record using secure encrypted means, and are immediately associated with the patient's name, medical record number, and other identifying characteristics. As a result of these images being stored, an automated request is then generated to a remote consultation organization in a remote country. There, a trained professional reviews the images of the patient's retina, and dictates a report detailing the pathologies noted in the patient's eyes. This report is also attached to the patient's electronic medical record, and the results are forwarded to the patient's doctors. If abnormalities are detected on this routine examination, a specialist examination can be obtained if needed. If no abnormalities are noted, the record can be used as a baseline measurement of the patient's retinal characteristics, which can then be used as a basis for comparison with later images.

A second example illustrates another possible usage scenario. A trained general practitioner examines the eyes of her patient. The instantly available digital images on the portable screen concern her; however, because she is a general practitioner, she is unsure if specialist consultation is warranted. To avoid the excess cost of sending the patient to see a specialist for a more detailed exam, she attaches the images to the patient's electronic medical record, and submits a request from to have the images reviewed by a retinal expert. Some time later, the report returned back indicates that the images she recorded are considered a variation of normal, and that no specialized examinations would be needed. In this way, the costs to the medical system and the patient have been reduced, and the patient is simultaneously receiving the expert opinion of a retinal specialist without the need for a separate formal evaluation.

The foregoing description is of exemplary and preferred embodiments employing at least in part certain teachings of the invention. The invention, as defined by the appended claims, is not limited to the described embodiments. Alterations and modifications to the disclosed embodiments may be made without departing from the invention. The meanings of the terms used in this specification are, unless expressly stated otherwise, intended to have ordinary and customary meaning and are not intended to be limited to the details of the illustrated structures or the disclosed embodiments.

What is claimed is:

1. A portable, direct ophthalmoscope for retinal examination comprising:
a handle;
a head connected with the handle, the handle enabling manually holding and positioning of the head near an eye being examined;
a light source arranged for shining light forwardly from the head, the light being transmitted toward the eye for illuminating the retina of the eye being examined when the head is positioned near and aimed toward the eye's pupil;
one or more imaging lens disposed within the head for magnifying an image of an object being illuminated by light from the light source, the object comprising the retina when the head is positioned near the eye being examined and is aimed at the pupil of the eye;
an image sensor optically coupled with the one or more imaging lenses for generating digital image data of the magnified image from the one or more imaging lenses.

2. The portable, direct ophthalmoscope of claim 2, wherein the image sensor is optically coupled for receiving all light being transmitted through the one or more imaging lenses, without any of the light being directed to an optical viewfinder.

3. The portable, direct ophthalmoscope of claim 2, wherein the image sensor is mounted within the head.

4. The portable, direct ophthalmoscope of claim 1, further comprising a display for displaying processes image data from the image sensor.

5. The portable, direct ophthalmoscope of claim 1, wherein the image sensor is mounted within an enclosure comprising at least in part the head; and wherein the portable direct ophthalmoscope further comprises a display for receiving processed image data generated by the images sensor and generate a display of the magnified image, the display being mounted to, or integrated with, the head in a position for viewing by a person manually holding the handle and aiming the head at an object of interest to be viewed on the display.

6. The portable, direct ophthalmoscope of claim 1, further comprising:
an optical viewfinder mounted on the head and coupled with the one or more imaging lenses for enabling a person holding the handle to view the magnified images;
an image capture device separate from the handle and the head, the image capture device having a housing, in which the image sensor is mounted, and a display mounted to the housing; and
a connector for releasably coupling the image capture device to the viewfinder for capturing the magnified image without splitting light coming from the one or more imaging lenses.

7. The portable, direct ophthalmoscope of claim 1, further comprising means within the head for automatically focusing the magnified image.

8. The portable, direct ophthalmoscope of claim 1, further comprising a programmable processor, the programmable processor being programmed for detecting an image of a retina within an image sensed by the image sensor.

9. The portable, direct ophthalmoscope of claim 8, wherein the processor is further programmed for automatically generating an alert in response to detection of an image of the retina.

10. The portable, direct ophthalmoscope of claim 8, wherein the processor is further programmed for automatically storing the image sensed by the image sensor in response to detection of the image of the retina.

11. The portable, direct ophthalmoscope of claim 8, wherein the processor is further programmed for automatically starting a predetermined, automated diagnostic process in response detection of an image of the retina.

12. The portable, direct ophthalmoscope of claim 1, further comprising means for transmitting the image to a remote display.

13. The portable, direct ophthalmoscope of claim 1, further comprising means for detecting spherical aberrations based on measured focal distances for focusing different parts of a retinal image captured by the image sensor.

14. The portable, direct ophthalmoscope of claim 1, further comprising means for measuring pulsations in vascular structures within a retina being examined based from video of the retina captured by the image sensor.

15. The portable, direct ophthalmoscope of claim 1, wherein the illumination is infrared illumination, and wherein the image sensor is sensitive to the infrared illumination.

16. The portable, direct ophthalmoscope of claim 1, further comprising one or more filters for selectively filtering light from the light source illuminating the retina, the filter or more filters chosen from the group of filters consisting of a visual acuity or a color blindness chart.

17. The portable, direct ophthalmoscope of claim 1, further comprising means for determining vision correction, the means for determining vision correction comprising an autofocus for focusing a beam of light on the retina of an eye, and a programmed processor for determining from the focusing the vision correction, if any.

18. The portable, direct ophthalmoscope of claim 1, further comprising means for stabling images generated from the image sensor.

19. A portable, direct ophthalmoscope for retinal examination comprising:
a handle;
a head connected with the handle, the handle enabling manually holding and positioning of the head near an eye being examined;
a light source arranged for shining light forwardly from the head, the light being transmitted toward the eye for illuminating the retina of the eye being examined when the head is positioned near and aimed toward the eye's pupil;
one or more imaging lens disposed within the head for magnifying an image of an object being illuminated by light from the light source, the object comprising the retina when the head is positioned near the eye being examined and is aimed at the pupil of the eye;
the head further comprising an optical viewfinder coupled with the one or more imaging lenses for enabling a person holding the handle to view the magnified images; and
a connector configured and adapted for releasably connecting to the head a separate image capture device and positioning the image capture device with respect to the optical viewfinder in order to enable capture of the magnified image by the image capture device, the connector including means for releasable attaching the image capture device to the connector;
wherein at least a portion of the connector is comprised of a rigid support member adapted for supporting the image capture device in a fixed position relative to the optical viewfinder when the image capture device is attached to the connector; and
wherein all of the light passing through the one or more imaging lenses is delivered to the optical viewfinder without being split.

20. The portable, direct ophthalmoscope of claim 19, further comprising an image capture device connected to the head with the connector, the image capture having a housing, in which an image sensor is mounted, and a display mounted to the housing for display images sensed by the image sensor.
